(19) United States
(12) Patent Application Publication
(10) Pub. No.: US 2004/0215057 A1
(43) Pub. Date: Oct. 28, 2004

(54) Portable, reusable visualization system

(76) Inventors: Parris S. Wellman, Hillsborough, NJ (US); Dale R. Schulze, Lebanon, OH (US); Gary W. Knight, West Chester, OH (US); Christopher J. Hess, Lebanon, OH (US); Kristopher Kundra, Lambertville, NJ (US); Michael F. Clem, Maineville, OH (US); Rudolph H. Nobis, Mason, OH (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/259,141

(22) Filed: Sep. 27, 2002

(51) Int. Cl. A61B 1/04

(52) U.S. Cl. 600/114; 600/160

(57) ABSTRACT

A portable endoscopic vessel harvesting system for use with a video display for retracting, viewing, and accessing tissue. An embodiment of the vessel harvesting system can include a first housing, an endoscope detachably mounted to the first housing, and a second housing detachably mounted to the endoscope, where the first housing configured to be disposable and the second portion configured to be reusable.

### FIELD OF THE INVENTION

The present invention relates generally to surgical instruments and video endoscopy. In particular, the present invention relates to an endoscopic instrument for harvesting a section of a blood vessel from a surgical patient.

### BACKGROUND OF THE INVENTION

The advantages of using endoscopic surgical procedures on patients are well known. Such procedures are minimally invasive, result in shortened hospital stays, more rapid recovery, less cosmetic damage, and lower overall costs compared to conventional “open” procedures.

An endoscopic procedure requires a way for the surgeon to visualize the operating site, which is generally at a location that the surgeon can not view using direct vision. In an endoscopic system, a surgical device is connected to a visualization system mounted in a “tower”. The tower generally includes a power source, a light source, an image processing unit, and a video display monitor. Towers occupy a lot of space in the operating room. Because space in the operating room is at a premium, the video display monitor is often not placed in an optimal position. Poor positioning of the monitor can make videoscopic surgery more difficult to perform because physical motions and viewed motions of the surgical tools may be reversed. Further, the location of the display monitor may require surgeons to turn their bodies or crane their necks to properly view the images, thereby increasing the time to perform, and the difficulty of, the procedure.

To overcome the problems of positioning the monitor and lack of space within the operating room, head-mounted displays (HMDs) or heads-up displays have been developed to permit the surgeon to view the operating space through a display that can be strapped onto the surgeon's head. See, for example, the product disclosed in U.S. Design Pat. No. D415,146 and commercially available from Vista. Those skilled in the art have used HMDs in conjunction with endoscopic procedures to displace the video display monitor either in connection with training or actual surgery. See, e.g., U.S. Pat. Nos. 6,309,345, 6,306,082, 6,120,433, 6,113,395, 5,846,185, and 5,749,830, all of which are incorporated herein by reference.

The advantage of the tower is that it permits the hospital to reuse the components housed in the tower again and again, as they are not located in the operating field and thus do not require sterilization. The tower components—a power source, light source, image processing unit, and video display monitor (if an HMD is not used)—are expensive compared with the cost of the tools used in the procedure and the access device used to deliver the image from the operation site to the monitor. Endoscopic vessel harvesting (EVH), or the surgical removal of section of a blood vessel from a surgical patient for use in another part of the patient's or another's body, is a complex procedure that would benefit from making the components of the devices and the visualization system more portable, yet cost-effective. By way of background, a conventional vessel harvesting device 1 is shown in FIGS. 1-3. Referring to FIG. 1, harvesting device 1 includes a hollow shaft 4 connected to a concave head piece 8 located at the distal end of shaft 4. Concave head piece 8 serves to provide the surgeon with workspace 6. Workspace 6 may be viewed via an endoscope 5, which is disposed within shaft 4 and includes a viewing lens 5a at the distal end of endoscope 5. The edge 8a of head piece 8 is used for dissecting the vessel from the surrounding tissue as shown in FIGS. 2 and 3. The device 1 may also have guide rails located on the underside of the device to permit access to workspace 6 with other devices, such as dissectors, ligation tools, and cutting tools.

The method of using device 1 to remove a vessel section is shown in FIGS. 2 and 3. Initially, an incision 3 is made and vessel 7 is located. Then, vessel 7 is dissected from the surrounding tissue 2 using the leading edge of the head piece 8 of the device 1 to separate tissue 2 from the vessel 7. At this time there is sufficient workspace 6 created around vessel 7 so that other instruments can be inserted into incision 3 via guide rails located on the underside of the device. These instruments include ligation tools for securing side branch vessels, a vessel dissector for performing a more complete dissection of the vessel, and laparoscopic scissors for the transection of both the side branch vessels 9 and the vessel 7 to be removed.

Further examples of endoscopic vessel harvesting systems are found in U.S. Pat. Nos. Re. 36,043, 6,206,823, 6,139,489, 5,968,066, 5,725,479 and 5,722,934, the disclosures of which are hereby incorporated by reference.

### SUMMARY OF THE INVENTION

The present invention provides devices and methods for visualizing endoscopic surgical procedures. In particular, the present invention relates to an endoscopic instrument for harvesting a section of a blood vessel from a surgical patient.

One preferred embodiment of the invention is a wearable, compact and portable video system. The system can include a housing having a first portion and a second portion. The first portion houses components or is connected to components that are disposable, while the second portion houses components or is connected to components that are reusable. In one embodiment, the first portion is simply a handle. An endoscope is detachably mounted to one or both of the first and second portions of the housing. Preferably the endoscope is slidably detachable to the first portion of the housing. An imaging unit is housed within the second portion of the housing in optical alignment with the viewing passageway of the endoscope. A light source is preferably housed within the second portion of the housing and can be detachably mounted to the endoscope. A power source, preferably a battery unit, is electrically connected to the imaging unit and the light source and is configured to be attachable to a practitioner. A display for displaying the signal transmitted by the imaging unit is configured to be attachable to the practitioner. Preferably, the display is an HMD.

The battery unit and display are all capable of being mounted on a belt, bandolier or backpack worn by the surgeon. Further, the light source and the imaging unit may be integrated with the battery unit into a compact unit, which can be mounted directly to the endoscope. Such a configuration does not require a large “footprint” in the operating room and does not require extensive set up by the surgical staff. The HMD provides optimal positioning of the display with respect to the surgeon. In addition, most if not all of the costly components are reusable, thereby reducing the cost of the procedure.

A further understanding of the nature and advantages of the invention and further aspects and advantages of the invention maybe realized by reference to the remaining portion of the specification and the drawings.

### BRIEF DESCRIPTION OF THE DRAWINGS

FIG. 1 is a perspective view of an endoscopic vessel harvesting device of the prior art.

FIG. 2 is a perspective view of a surgeon and an endoscopic vessel harvesting device of the prior art harvesting a vein located in a person's leg.

FIG. 3 is an enlarged perspective view of the endoscopic vessel harvesting of FIG. 1 inserted into a patient during a procedure to harvest a vein.

FIG. 4 is a front-view schematic of a surgeon utilizing the endoscopic video system according to the invention.

FIG. 5 is a rear-view schematic of a surgeon utilizing the endoscopic video system of FIG. 4.

FIGS. 6A-6C are perspective views of an endoscopic device shown in three stages of assembly.

FIG. 7 is a partial cross section of the proximal end of an endoscopic device depicting the internal components of the second portion of the housing.

FIG. 8 is a top plan view of an endoscopic device according to a preferred embodiment of the invention.

FIG. 9 is a side view of the endoscopic device of FIG. 8.

### DETAILED DESCRIPTION OF THE SPECIFIC EMBODIMENTS

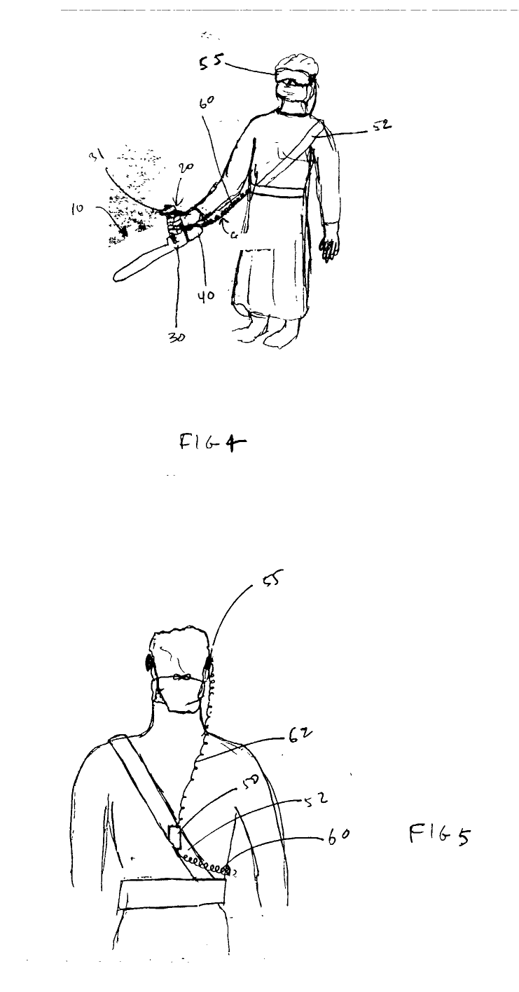

Referring to the figures, FIGS. 4 and 5 show a schematic of the videoscopic endoscopic vein harvesting system being used by a surgeon, indicated generally as reference numeral 10. The system 10 comprises a vein harvesting device 20 that includes a first portion 30, and a second portion 40 slidably connected to the first portion 30. System 10 also includes an endoscope 70 which is preferably detachably mounted to either first portion 30 or second portion 40 or both. The first portion 30 includes a handle 31 for the surgeon to grasp while manipulating the device. In a preferred embodiment, first portion 30 functions as a handle and as a base from which. The second portion 40 houses a light source and video imaging unit (described herein) and is connected to power source 50 by a first cable 60 and to a head mounted display (HMD) 55 by a second cable 62. The power source 50 and HMD 55 are preferably configured to be worn on the surgeon's body. In a preferred embodiment, the power source 50 is a battery pack attached to a belt or bandolier 52 or a backpack. The HMD 55 is preferably a pair of commercially available video glasses, such as the Sony Glasstron video viewing glasses. The power source 50 may be a backup source of power or the primary source of power. The power source 50 and belt 52 are kept out of the sterile field where they can be reached by a non-sterile circulating nurse if a battery replacement is required during the procedure. FIG. 5 shows one mounting arrangement for the power source 50 on belt 52.

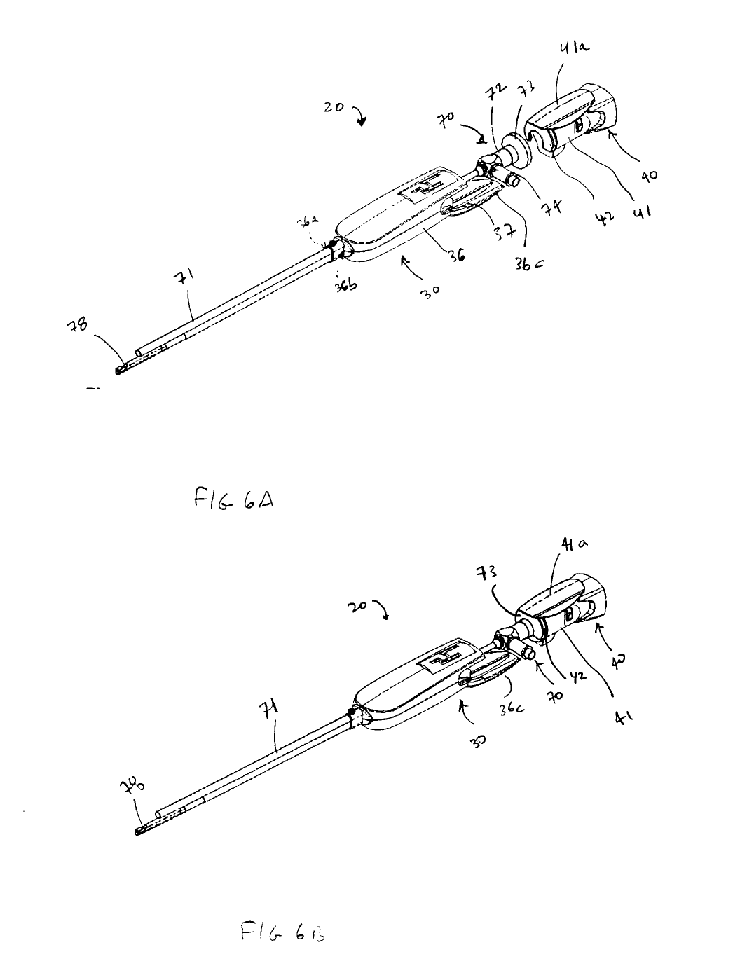

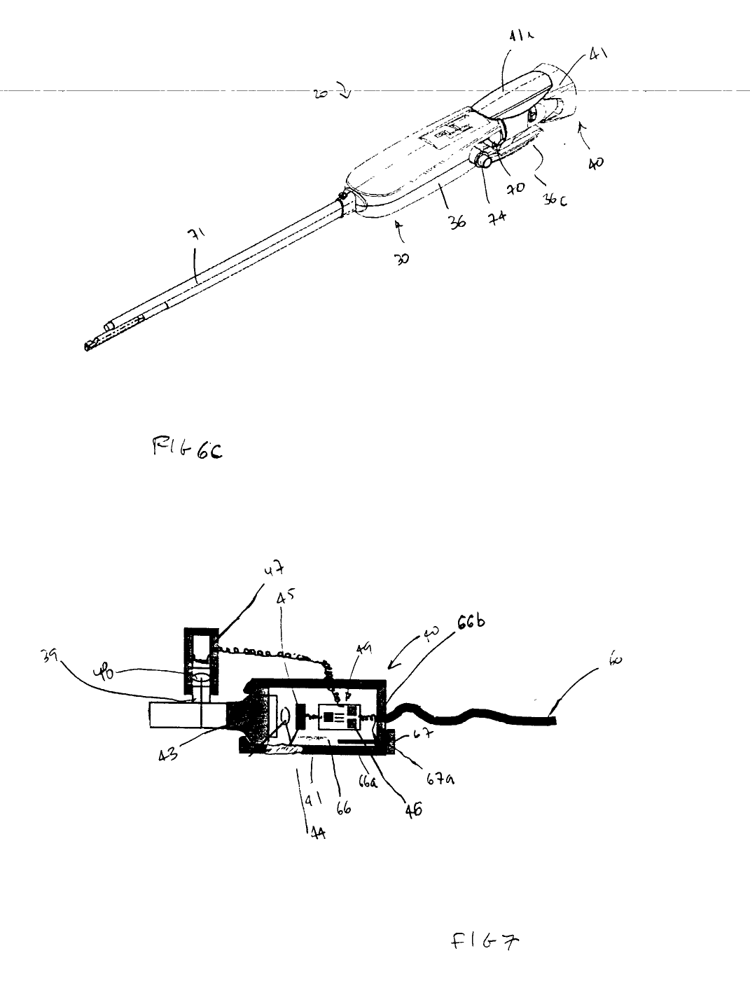

FIGS. 6A-6C disclose perspective views of one embodiment of the endoscopic vein harvesting device 20 in three stages of assembly. FIG. 6A depicts the device 20 partially disassembled, with an endoscope 70 partially disposed within the first portion 30, and a second portion 40 shown disengaged from the endoscope 70. FIG. 6B depicts a second stage of assembly, where the endoscope 70 is attached to the second portion 40. FIG. 6C depicts a third stage of assembly, where endoscope 70 is captured between the first portion 30 and the second portion 40.

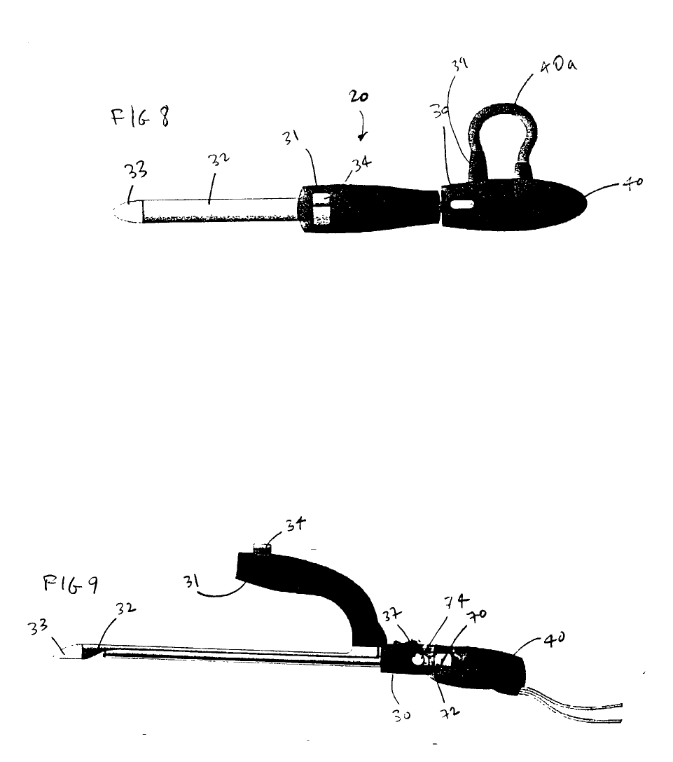

First portion 30 shown in FIGS. 6A-6C is a simplified configuration depicted without the handle or retractor (which can be similar to the handle and retractor depicted in the embodiments of FIGS. 8 and 9). First portion 30 includes a first housing 36 having a recess 37 and at least one lumen sized to accommodate endoscope 70 and an endoscopic tool 78. Endoscopic tool 78 can include bipolar scissors, a bipolar clamp, a coag-cut device, monopolar RF scissors or a harmonic scalpel, for example. Those skilled in the art will recognize other endoscopic tools that may be used in conjunction with this device. First portion 30 may also include actuators for controlling endoscopic tool 78.

Preferably first housing 36 has a first lumen 36a sized to accommodate the endoscope 70 and a second lumen 36b sized to accommodate the endoscopic tool 78. In another embodiment, first housing can be configured to accommodate only an endoscope and an endoscopic tool can be mounted to or disposed on the endoscope. For example, a transparent optical dissector tip can slidably engage the distal end of the endoscope, thereby obviating the need for a second lumen or a larger first lumen for accommodating an endoscopic tool.

Endoscope 70 is a conventional endoscope having a tube 71 and a body 72 attached to tube 71. Body 72 includes a mating flange 73 for mating with second portion 40 and a mating post 74 for mating with first portion 30. A viewing passageway extends longitudinally through body 72 and tube 71 for permitting illumination of the operating space distal to endoscope 70 by a light source and for permitting an image viewed at the distal end of endoscope 70 to be transmitted to the proximal end of endoscope 70. The tube 71 is preferably formed of a rigid material, for example a medical grade stainless steel, or a rigid plastic. Recess 37 of first housing 36 is configured to slidingly accept the mating post 74 of endoscope 70.

Second portion 40 includes a mating portion 42 configured to accept mating flange 73 of endoscope 70. As is shown in FIG. 6B, when mating flange 73 is disposed at least partially within mating portion 42, second portion 40 is attached to endoscope 70. In this configuration, when endoscope 70 is moved distally relative to first portion 30, endoscope 70 serves to retain first portion 30 and second portion 40 in their mated configuration (as is shown in FIG. 6C). First portion 30 and/or second portion 40 may include a locking assembly to releasably lock first portion 30 and second portion 40 to endoscope 70 or to each other. The first portion 30 and the second portion 40 are preferably formed of a thermoplastic.

As described above, endoscope 70 and first housing 36 are configured to matingly engage and endoscope 70 and second housing 41 are configured to matingly engage. The embodiment described demonstrates two ways in which this may be accomplished: a post and recess or a flange and mating portion. Those skilled in the art can devise numerous ways to accomplish the objective of engaging endoscope 70 with first housing 36 and second housing 41, including latching one to the other by using an elastomeric press fit, a clamp, threaded portions, a locking feature, hooks and loops, magnets or other means known to those skilled in the art. First housing 36 and second housing 41 need not have a separate mechanism for holding them together when the surgeon uses the device. For example, referring to FIGS. 6A-6C, first housing 36 can include a bottom surface 36c and second housing 41 can include a top surface 41a that are designed such that, when first portion 30 and second portion 40 are matingly engaged, bottom surface 36c rests in the surgeon's four fingers and top surface 41a is contacted by the surgeon's thumb. Thus, first portion 30 and second portion 40 can simply be held together in the surgeon's hand while the surgeon is using the device. Preferably, first housing 36, endoscope 70 and second housing 41 are configured such that they securely engaged with one another when the surgeon uses the instrument such that one element does not inadvertently slide with respect to another during the procedure. Referring to FIG. 7, a schematic depiction of the components of second portion 40 are shown. The primary components of this system include a light source 47 with an optional first focusing lens 48, an imaging unit 49 with a second focusing lens 44, and a focusing stage 66 on which the second lens 44 rides. When second portion 40 is matingly engaged with endoscope 70, the viewing passageway is optically aligned with the light source 47 and first focusing lens 48.

The light source 47 can be a krypton, halogen, or xenon bulb, and is coupled to a light port 39 of a standard endoscope. Light port 39 can also be attached to first portion 30. The light from light source 47 may optionally be passed through a focusing lens prior to being directed into the viewing passageway of the endoscope. The light port 39 typically contains a mirror or prism that directs the light generated by light source 47 through optical fibers or another light transmission means to the distal end of endoscope 70. The light source 47 can be of any source, including incandescent, solid state (light emitting diodes), fluorescent, white LEDs (phosphor based or rare earth), or a composite source made from red, green and blue LEDs, for example. The imaging unit 49 can include any necessary processing electronics for image formation or translation to an appropriate communication format, such as NTSC or PAL, for example.

Second portion 40 includes a second housing 41 for housing second focusing lens 44 and an imaging unit 49. Second housing 41 has a window 43 in its distal end that permits images to be transmitted from endoscope 70 to the second focusing lens 44. Window 43 is formed of a substantially transparent medical grade material, such as polycarbonate. Imaging unit 49 includes an imaging chip 45 optically aligned with the first focusing lens 44, and a power conditioning/encoding board 46. A single achromatic lens 44 is shown for focusing the output of the endoscope 70 onto the imaging chip 45. Alternatively, a second focusing lens 44 can include one or more lenses of the same or various types, including plastic injection molded diffractive optical lenses that can correct chromatic aberration. Further, second focusing lens 44 can be used in conjunction with or replaced by a parabolic mirror that focuses the output of the lamp or LED light source onto the end of the endoscope fiber or the lens can be formed as an integral portion of the light source.

The position of second lens 44 relative to the imaging chip 45 is preferably adjustable, and can be controlled by moving the focusing stage 66 in a longitudinal direction. This may be accomplished by using a lead screw 66a that can either be directly coupled to a knob 67 through the second housing 41, or through a magnetic coupling. The magnetic coupling includes a first magnet 67a housed in knob 67 and a second magnet 66b attached to the end of lead screw 66a proximal second housing 41. First magnet 66a and second magnet 67b are configured to have a magnetic field of a strength that causes one to move when the other moves. In this way, rotating knob 67 rotates first magnet 67a, which in turn rotates second magnet 66b and lead screw 66a, thereby moving focusing stage 66 with respect to lead screw 66a and adjusting the distance between second lens 44 and imaging chip 45. Such a magnetic coupling allows second housing 41 to be sealed, thereby permitting second housing 41 and second portion 40 to be resterilized upon completion of a procedure.

The image produced by the imaging chip 45 is transmitted to the power conditioning/encoding board 46, which processes the signal and transmits the signal via power and signal cable 60 to the HMD 55. The imaging chip 45 is preferably a solid state imager and may be a charge coupled device (CCD), a complementary metal oxide semiconductor (CMOS), a photo-multiplier tube (PMT) or other light-sensitive, solid-state imager. Preferably, the imaging device would be a CCD “single chip camera” because of the ease with which it may be implemented and its high sensitivity and video quality. Power conditioning electronics and light intensity regulating electronics may be included on a separate board 46 within second housing 41 or may be part of the same board as the imaging chip 45.

Second portion 40 preferably houses most of the costly elements of system 10, such as the light source and focusing lenses. As a result, second portion 40 is preferably designed to be reusable. Second housing 41 and window 43 are formed of materials known in the art to be resterilizable, such as polycarbonate and other medical grade plastics. Second housing 41 is preferably sealed to endure a resterilization process by ultrasonically welding the housing portions together or by using a similar means to create a gas- and water-impermeable seal. The resterilization process can consist of sterilization using ETO, a cold chemical process or a hydrogen peroxide plasma, for example. Gamma irradiation is not typically an appropriate sterilization process as the chip could be damaged or destroyed during such a process. While second portion 40 is preferably reusable, it may also be disposable depending on cost considerations.

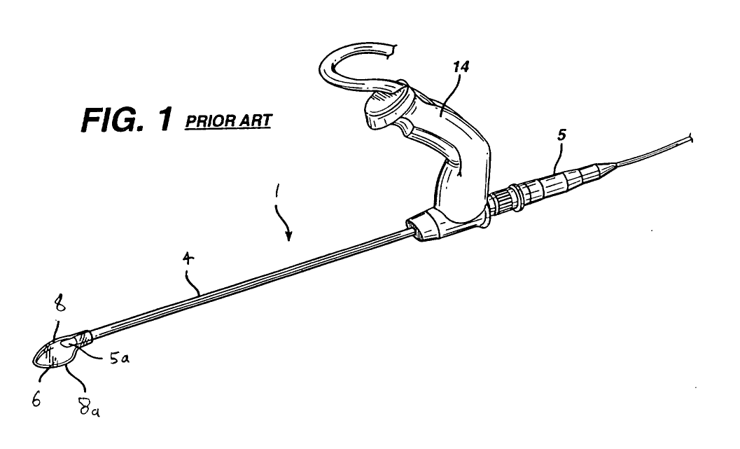

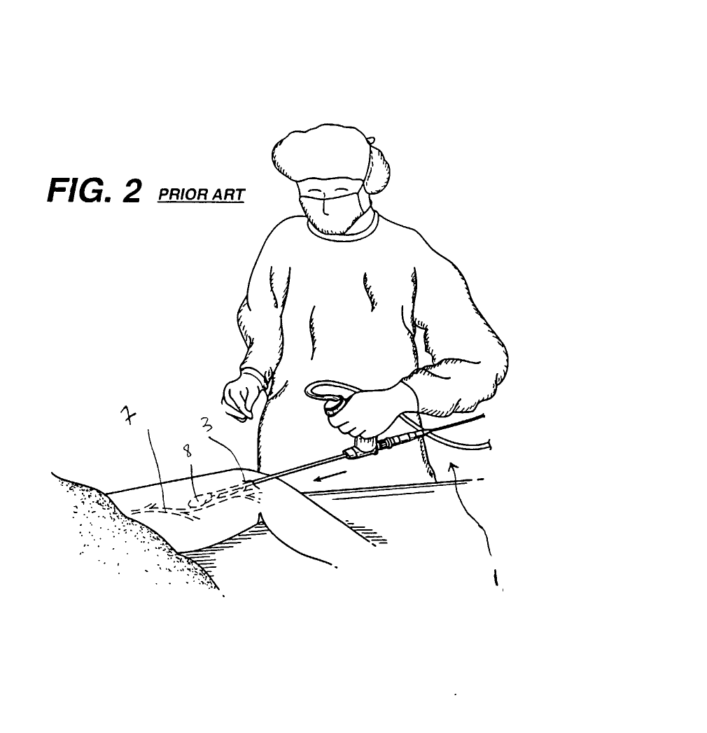

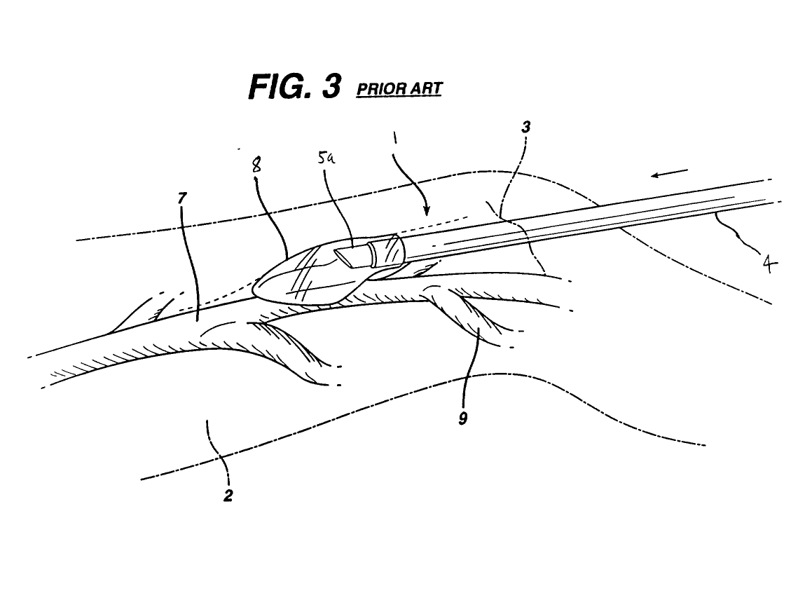

Referring to FIGS. 8 and 9, a preferred embodiment of the device 20 is depicted in plan and side views, respectively, with similar parts numbered similarly. Device 20 of FIGS. 8 and 9 provides a low profile device designed to be used like the prior art device 1 of FIGS. 1-3. In the prior art embodiment, however, the harvesting device 1 consists of an endoscope 5 that is simply passed through a hollow shaft 4. Further, the image processing unit and the light source are external to the prior art device 1. In contrast, 20 device 20 of FIGS. 8 and 9 includes a first portion 30 through which endoscope 70 is passed, and a second portion 40, which mates with first portion 30 and endoscope 70 and houses the image processing unit and the light source.

Referring to FIG. 9, device 20 includes a first portion 30 having a retractor shield 32 extending distally therefrom. Shield 32 may be a hollow shaft or simply an arcuate, elongate section, but in any case shield 32 is configured to separate tissue from the device 20 and any other devices that may be passed beneath shield 32. A concave headpiece 33 is connected to the distal end of shield 32 and serves to provide the surgeon with workspace. Headpiece 33 is preferably formed of a substantially transparent medical grade material, such as polycarbonate. The workspace created by headpiece 33 may be viewed with 30 endoscope 70, which is generally disposed beneath or within shield 32. In a preferred embodiment, first portion 30 includes a lumen through which endoscope 70 is passed. First portion 30 also includes a handle 31 that may include one or more actuators 34 for operating device 20.

As in the embodiments described above, endoscope 70 includes a tube (hidden) and a body 72 attached to the tube. Body 72 includes a mating post 74 for mating with first portion 30. A viewing passageway extends through body 72 and the tube for permitting illumination of the operating space formed by headpiece 33 by a light source and for permitting an image viewed within the operating space to be transmitted to the proximal end of endoscope 70. A recess 37 formed in first portion 30 is configured to slidingly accept the mating post 74 of endoscope 70. Second portion 40 is configured to matingly engage endoscope 70 and first portion 30. Second portion 40 may include light power cable 40a for detachably providing power to light port 39. Power cable 40a if preferably clad in a non-permeable material known to those skilled in the art.

While the endoscopic system described above includes a retractor that establishes a working space mechanically with a headpiece 33 and retractor shield 32, a device that establishes a working space by using insufflation, such as the device described in U.S. Pat. No. 6,432,044, which is hereby incorporated by reference, can include a three-part system having a disposable portion, an endoscope and a reusable portion.

The video system described herein is useful for a number of different medical procedures. These procedures include endoscopic vessel harvesting, diagnostic and therapeutic hysteroscopy, endoscopic orthopedic surgery, laparoscopy, thoracoscopy and video assisted cardiac surgery. The device also has varied non-medical applications. These include video borescope examination of engine cylinders and other remote visualization applications.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected.

What is claimed is:

1. A portable endoscopic vessel harvesting system, comprising:
a housing having a first portion and a second portion, the first portion being disposable and the second portion being reusable;
an endoscope detachably mounted to at least one of the first portion and the second portion of the housing, the endoscope having a viewing passageway;
an imaging unit disposed within the second portion of the housing, the imaging unit optically aligned with the viewing passageway;
a light source detachably mounted to one of the endoscope and the second portion, the light source configured to provide light to the distal end of the endoscope;
a power source electrically connected to the imaging unit and light source, the power source configured to be attachable to a practitioner; and
a display for displaying the signal transmitted by the imaging unit, the display configured to be attachable to the practitioner.

2. The system of claim 1, wherein the first portion and the second portion are configured to be slidably detachable to the endoscope.

3. The system of claim 1, wherein the display is a head-mounted display configured to be worn on the practitioner's head.

4. The system of claim 1, wherein the power source is a battery unit configured to be worn by the practitioner on his or her belt.

5. The system of claim 1, wherein the imaging unit is an imaging microchip and a power conditioning and encoding board.

6. The system of claim 5, wherein the imaging microchip is one of a charge coupled device, a complementary metal oxide semiconductor and a photo-multiplier tube.

7. The system of claim 1, comprising a focusing lens disposed between the endoscope and the imaging unit, the focusing lens disposed within the second portion of the housing.

8. The system of claim 1, wherein the light source is disposed within the second portion of the housing.

9. The system of claim 1, comprising a focusing lens disposed between the light source and the proximal end of the endoscope.

10. The system of claim 1, wherein the light source is one of incandescent, solid state, fluorescent and fiber optic.

11. A portable videoscopic surgical system, comprising:
a housing having a handle and a second portion, the second portion detachably connected to the handle;
an endoscope positionable within the housing;
an imaging unit disposed within the second portion of the housing, the imaging unit optically aligned with the endoscope, the imaging unit configured to provide an output signal;
a light source detachably mounted to the endoscope;
a power source electrically connected to the imaging unit, the power source configured to be worn by a practitioner; and
a head's up display, configured to be worn on the practitioner's head, for receiving the output signal from the imaging unit and displaying the image viewed at the distal end of the endoscope.

12. An endoscopic vessel harvesting system, comprising:
a handle;
an endoscope detachably mounted to the handle; and
a second housing detachably mounted to one of the endoscope and the handle, the handle configured to be disposable and the second portion configured to be reusable.

13. The endoscopic vessel harvesting system of claim 12, wherein the endoscope is configured to matingly engage with both the handle and the second housing.

14. The endoscopic vessel harvesting system of claim 12, wherein the endoscope includes a mating flange at a proximal end and the second housing includes a mating portion configured to matingly engage the mating flange.

15. The endoscopic vessel harvesting system of claim 12, wherein the endoscope includes a mating post at a distal end and the handle includes a recess configured to matingly engage the mating post.

16. The endoscopic vessel harvesting system of claim 12, comprising:
a lens disposed within the second housing;
a focusing stage disposed within the second housing and attached to the lens;
a first magnet attached to the focusing stage and disposed within the second housing; and
a second magnet disposed outside the second housing, the second magnet configured to rotate the first magnet when the second magnet is rotated.

17. The endoscopic vessel harvesting system of claim 16, comprising an imaging chip disposed within the second housing aligned with the lens.

18. The endoscopic vessel harvesting system of claim 17, wherein the focusing stage includes a lead screw extending therefrom, the first magnet being attached to the free end of the lead screw, the second magnet being attached to the outer surface of the second housing opposite the first magnet.
